(19) United States
(12) Patent Application Publication
(10) Pub. No.: US 2024/0269091 A1
(43) Pub. Date: Aug. 15, 2024

(54) IDEBENONE IN THE TREATMENT OF DRUG RESISTANT EPILEPSY

(71) Applicant: Katholieke Universiteit Leuven, Leuven (BE)

(72) Inventors: Karin THEVISSEN, Bierbeek (BE); Bruno CAMMUE, Alsemberg (BE); Peter DE WITTE, Kessel-Lo (BE); Lieven LAGAE, Oud-Heverlee (BE); Annelii NY, Mol (BE)

(21) Appl. No.: 18/568,058

(22) PCT Filed: Jun. 7, 2022
(86) PCT No.: PCT/EP2022/065422

§ 371 Date: Dec. 7, 2023

(30) Foreign Application Priority Data
Jun. 7, 2021 (EP) 21178051.5

(51) Int. Cl. A61K 31/122; A61P 25/08

(52) U.S. Cl. CPC A61K 31/122; A61P 25/08

(57) ABSTRACT

The invention relates to idebenone for use in the prevention or treatment neuroinflammation and seizures in an epilepsy patient.

### FIELD OF THE INVENTION

The invention relates to treatment of epilepsy.

### BACKGROUND OF THE INVENTION

Epilepsy is among the most common severe neurological conditions, affecting more than 70 million people worldwide [Bialer et al. (2017) Epilepsia 58, 181-221; Jaspars et al. (2016) J. Marine Biol. Ass. UK 96, 151-158; Kong et al. (2010) Drug Discov Today 15, 884-886]. It is characterized by an enduring predisposition of the brain to generate epileptic seizures, with neurobiologic, cognitive, psychological, and social consequences [Sakai and Swanson (2014) Nat Prod Rep 31, 273-309]. The treatment of epilepsy consists mostly of pharmacotherapy with antiseizure drugs (ASDs) to control seizures [West and Crawford (2016) Planta Med 82, 754-760]. Despite considerable efforts, current ASDs fail to control the seizures of 30% of patients due to drug-resistance [Howe et al. (2013) Nature 496, 498-503].

There remains a need for therapies for drug resistant epilepsy.

Idebenone is described as a medicament for the treatment of Alzheimer's disease [EP0629400].

Idebenone is described as a medicament for the treatment and/or prophylaxis of weakness and/or loss of skeletal muscle tissue and/or cardiomyopathy associated with a muscular dystrophy. [EP1861080]

WO2010124713 describes idebenone in treating and/or preventing primary progressive multiple sclerosis (PP-MS).

Idebenone has been used in an acute pilocarpine-induced seizure model in rats, as a preventive treatment, 3 days prior to the pilocarpine injection) [Ahmed (2014) Neurochem Res. 39, 394-402] The administration of idebenone provides neuroprotection.

### SUMMARY OF THE INVENTION

Idebenone increases activity of the enzyme PHGDH, which is necessary for correct brain functioning as well as for polarizing macrophages/microglia toward anti-inflammatory status. PHGDH malfunctioning has been linked to drug resistant epilepsy. The efficacy of idebenone in a preclinical model for drug resistant epilepsy as well as in a mouse model for inflammation has been demonstrated.

The present invention relates to idebenone as an anti-epileptic treatment for drug resistant epilepsy, thereby alleviating neuro-inflammation.

The present invention relates to idebenone for treating and preventing neuro-inflammation.

The invention relates to idebenone for use in the prevention or treatment of a drug resistant epilepsy.

One aspect of the invention relates to idebenone for use in the treatment or prevention of a seizures in a drug resistant epilepsy.

In an embodiment hereof idebenone is for use in the treatment or prevention of a seizures in a drug resistant epilepsy and neuroinflammation associated with epilepsy

Another aspect of the invention is idebenone for use in the treatment or prevention of neuroinflammation in an epilepsy patent.

In an embodiment thereof idebenone is for use in the treatment or prevention of neuroinflammation in an epilepsy patient and in the treatment and prevention of a seizure in said patient.

In specific embodiments, the epilepsy is a drug resistant epilepsy.

An another aspect of the invention relates to methods of treating or preventing neuroinflammation associated with epilepsy, comprising the step of administering to an epilepsy patient a therapeutic effective amount of idebenone. In an embodiment, the method further treats or prevents an epileptic seizure in said patient. In specific embodiments, the epilepsy is a drug resistant epilepsy.

In all the above aspects and embodiments, drug resistant epilepsy in an epilepsy resistant against two or more drugs selected from the group consisting of valproate, carbamazepine, levetiracetam, lamotrigine, topiramate, briveracetam, lacosamide, perampanel, and phenobarbital.

### DESCRIPTION OF THE INVENTION

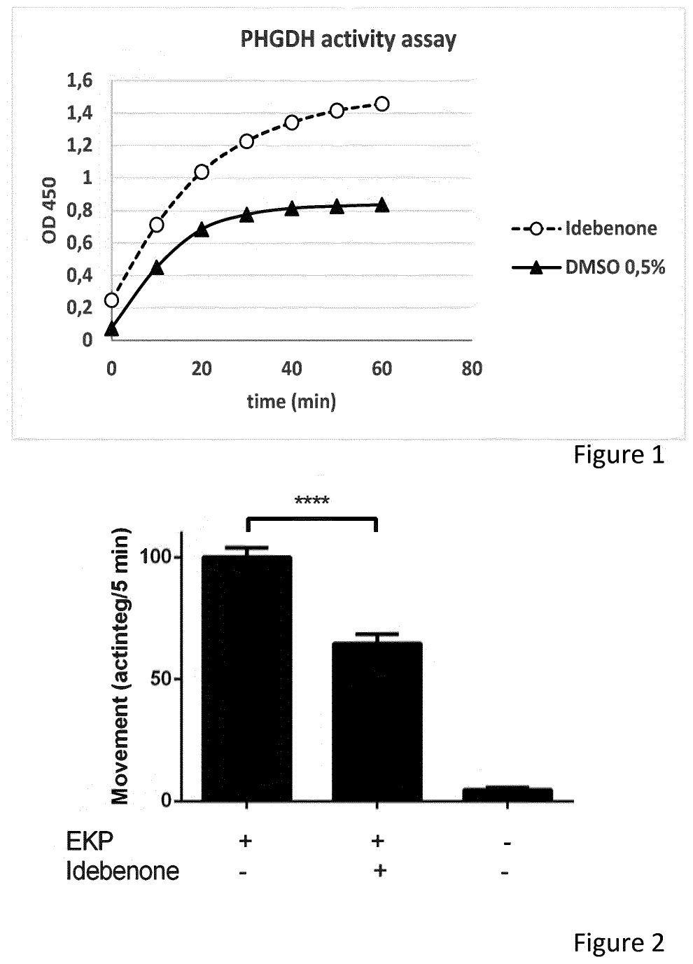

FIG. 1. PHGDH activity over time in presence of 25 UM of Idebenone; DMSO background for all experiments was 0.5%.

FIG. 2. Activity profile of 8.5 μM idebenone after 300 μM EKP exposure. Locomotor activity was quantified using ZebraLab™ software (Viewpoint, Lyon, France) and expressed in mean “actinteg” units per 5-min+/−SEM during a 30 min recording interval as indicated above. Statistical analysis: one-way ANOVA with Dunnett's multiple comparison test. A statistical difference is indicated by: ****p<0.0001. For each condition n=10 larvae were used and the experiment was performed three times (n total=30 per condition).

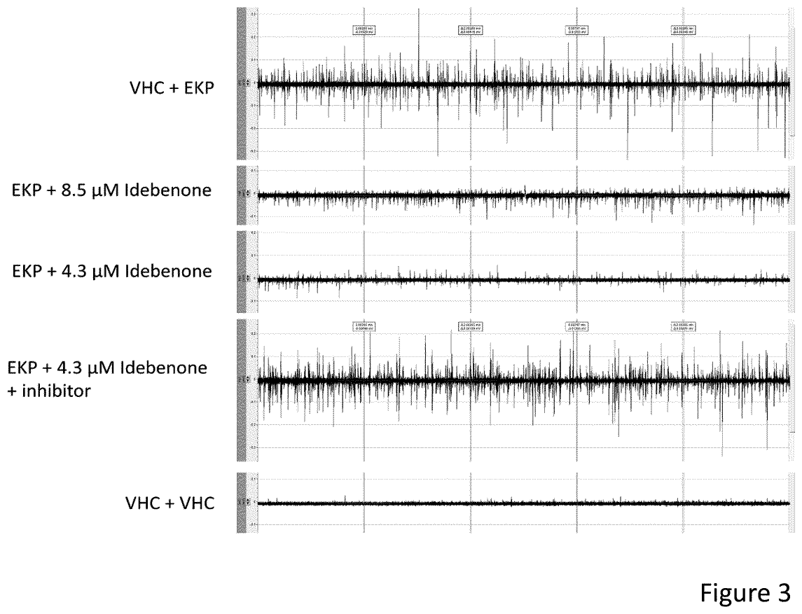

FIG. 3. Representative LFP recordings of zebrafish in the absence (VHC) or presence of EKP, treated with 8.5-4.3 μM idebenone or untreated (VHC), in the presence or absence of a specific PHGDH inhibitor CBR-5884 [Mullarky et al. (2016) Proc Natl Acad Sci USA 113, 1778-1783].

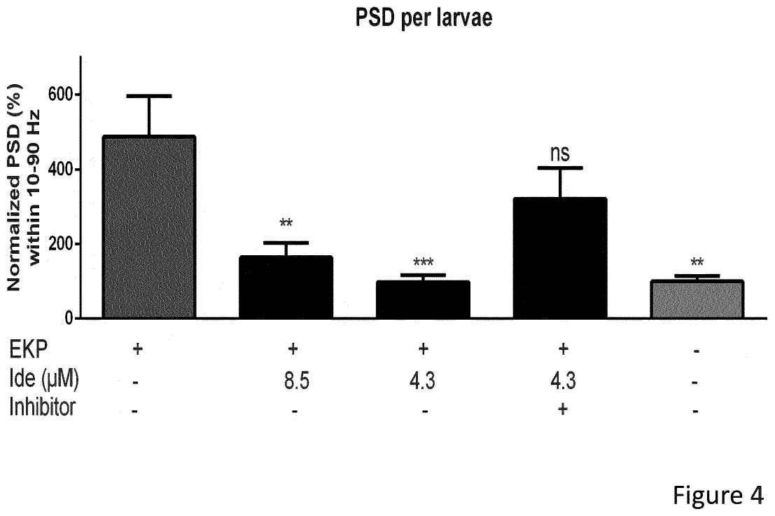

FIG. 4: Normalized PSD per larvae for the different conditions. Number of epileptic events within a 10-min recording (mean±SEM.). Number of recordings analysed were: VHC (n=9), EKP (n=15), 8.5 μM idebenone (Ide) (n=10), 4.3 μM idebenone (n=10), 4.3 μM idebenone+inhibitor (n=9). PHGDH-inhibitor CBR-5884 was used. Statistical differences as compared to the EKP-only treated larvae were assessed by the following statistical analysis: one-way ANOVA with Dunnett's multiple comparison test. A statistical difference is indicated by: **p<0.01, ***p<0.001 and ****p<0.0001.ns=not significant.

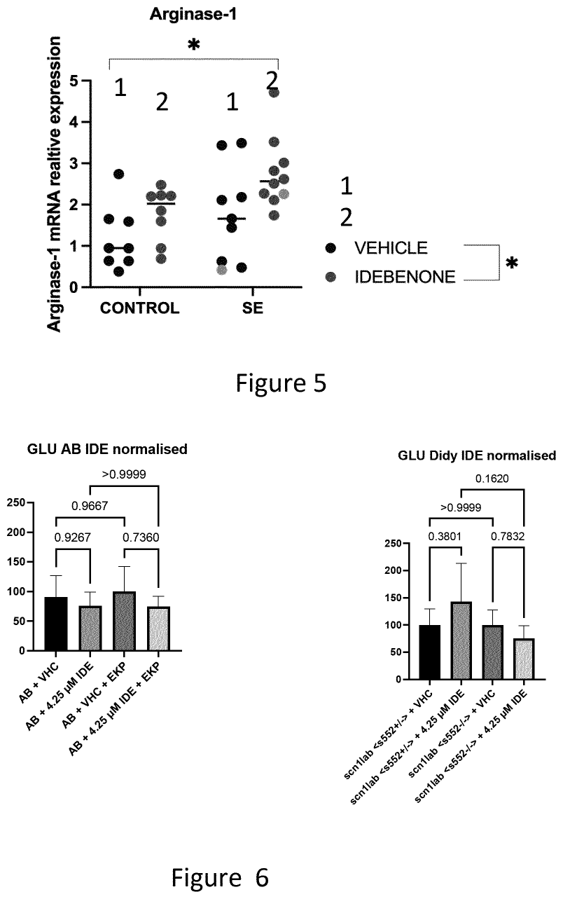

FIG. 5: Idebenone increases the expression of a key marker for the M2 microglia phenotype, Arginase 1.

FIG. 6: Analysis of glutamate levels via LCMSMS in zebrafish heads treated with idebenone (IDE) vehicle (VHC).

Idebenone has the chemical name 6-(10-hydroxydecyl)-2,3-dimethoxy-5-methyl-1,4-benzoquinone. Idebenone is a synthetic analogue of coenzyme Q10 (CoQ10), the vital cell membrane antioxidant and essential constituent of the adenosine-triphosphate (ATP)-producing mitochondrial electron transport chain (ETC). Idebenone has the ability to operate under low oxygen tension situations. Due to its ability to inhibit lipid peroxidation, idebenone protects cell membranes and mitochondria from oxidative damage (Zs.-Nagy (1990) Arch. Gerontol. Geriatr. 11, 177-186). Its antioxidant properties protect against cerebral ischemia and nerve damage in the central nervous system. Idebenone also interacts with the ETC, preserving ATP formation in ischemic states.

The toxicity of idebenone is very low; for example, its LD50 as an index of acute toxicity exceeds 10,000 mg/kg in male and female mice, exceeds 10,000 mg/kg in male rats and is about 10,000 mg/kg in female rats.

Possible modes of administration are oral, i.p., i.v., i.m., i.e, parenteral, intranasal and transdermal, whereas the oral administration is the typical mode of administration.

EP2051706 describes methods for transmucosal administration of idebenone. Further details on methods of administration and formulations are disclosed in patent applicants of Santhera (EP 1378753; EP1861080; EP2004176; EP2051706; EP2108366; EP2424513; EP2804596; EP3488846)

“Drug-resistant epilepsy (DRE)” is defined by Kwan et al. (2010) Epilepsia 52, 1069-1077, as “failure of adequate trials of two tolerated and appropriately chosen and used antiepileptic drugs (AED schedules) (whether as monotherapies or in combination) to achieve sustained seizure freedom.”

A non-exhaustive list of anti-epileptic compounds includes Paraldehyde; Stiripentol; Barbiturates (such as Phenobarbital, Methylphenobarbital, Barbexaclone; Benzodiazepines (such as Clobazam, Clonazepam, Clorazepate, Diazepam Midazolam and Lorazepam); Potassium bromide; Felbamate; Carboxamides (such as Carbamazepine Oxcarbazepine and Eslicarbazepine acetate); fatty-acids (such as valproic acid, sodium valproate, divalproex sodium, Vigabatrin, Progabide and Tiagabine); Topiramate; Hydantoins (such as Ethotoin, Phenytoin, Mephenytoin and Fosphenytoin); Oxazolidinediones (such as Paramethadione Trimethadione and Ethadione); Beclamide; Primidone; Pyrrolidines such as Brivaracetam Etiracetam Levetiracetam; Seletracetam; Succinimides (such as Ethosuximide, Phensuximide and Mesuximide); Sulfonamides (such as Acetazolamide, Sultiame Methazolamide and Zonisamide); Lamotrigine; Pheneturide; Phenacemide; Valpromide; Valnoctamide; Perampanel; Stiripentol; Pyridoxine.

Specific types of “drug resistant epilepsy” are an epilepsy resistant against two or more drugs selected from the group consisting of valproate, carbamazepine, levetiracetam, lamotrigine, topiramate, briveracetam, lacosamide, perampanel, and phenobarbital.

“Treatment” relates to any improvement in the disease such as shorter periods of seizures, less severe seizures, less frequent seizures.

Neuroinflammation refers to inflammation (typically chronic) of nervous tissue, for example in the brain.

As most of these drugs have been reported to be effective in the pilocarpine model, anti-epileptic activity in this model does not unequivocally translate to an AED with the potential to treat drug resistant epilepsy [Bo-Qiang et al. (2018) Pharmazie 73, 207-212; Leclercq & Kaminski (2015) Epilepsy Behav. 49, 55-60; Wang et al. (2019) Brain Res. 1712, 1-6; Shishmanova-Doseva et al. (2020) Folia Med (Plovdiv) 62(4), 723-729; Ge et al. (2020) Curr Neurovasc Res. 17, 354-360].

The L-serine biosynthetic enzyme 3-phosphoglycerate dehydrogenase (PHGDH) catalyses the first and rate-limiting step of de novo serine synthesis [reviewed in Grant (2018) Front Mol Biosci. 5, 110].

Evidence to increase activity of PHGDH as a novel treatment option against (drug resistant) epilepsy stems from different research lines:

PHGDH activity is linked with brain function. L-serine (synthesized via PHGDH activity) is a key rate-limiting factor for maintaining steady-state levels of D-serine in the adult brain. Hence, L-serine availability in mature neuronal circuits determines the rate of D-serine synthesis in the forebrain and controls N-methyl-d-aspartate (NMDA) receptor function at least in the hippocampus. Hippocampal NMDA receptor is a key player in the generation of seizures. Moreover, the enzyme following PHGDH for de novo serine biosynthesis, PSAT1, utilizes glutamate. Glutamate homeostasis is crucial for correct brain functioning; glutamate is regarded as one of the most important pro-convulsant neurotransmitters. By increasing activity of PHGDH, it is proposed that the activity of the downstream enzyme PSAT1 will be increased as well. As PSAT1 utilizes glutamate in its reaction, increasing the activity of PHGDH might reduce excess levels of glutamate.

PHGDH malfunctioning/deficiency is associated with (drug resistant) epilepsy. In humans, PHGDH deficiencies have been reported; the hallmarks of PHGDH deficiency are microcephaly of prenatal onset, severe psychomotor disability, early intractable seizures (of various type), and progressive spasticity. PHGDH deficiencies can be subdivided in two severe recessive phenotypes: classical PHGDH deficiency [Tabatabaie et al. (2011) J Inherit Metab Dis. 34, 181-184] and Neu-Laxova syndrome type 1 (NLS1) (Online Mendelian Inheritance in Man (OMIM) 256520; no residual PHGDH activity). NLS1 patients show severe, early onset, drug resistant epilepsy [Poli et al. (2017) Am J Med Genet. 173, 1936-1942]. Moreover, mice with reduced PHGDH expression, induced by a diet resulting in fatty liver disease, have a severe predisposition for development of seizures, more specifically increase seizure episodes and decreased seizure thresholds [Sim et al. (2020) Metabolism 102, 154000; Aksoy et al. (2014) Neurol Sci. 35, 1441-1446].

Upregulation of the expression of various genes encoding enzymes involved in de novo serine biosynthesis (including PHGDH) can be achieved by the ketogenic diet, which is an effective way to treat drug resistant epilepsy [Vazquez (2020) bioRxiv] PHGDH activity is linked to anti-inflammatory action. PHGDH has been identified as a key enzyme for steering macrophage polarization towards an anti-inflammatory M2 state [Wilson et al. (2020) Cell Rep. 30, 1542-1552]. Hence, PHGDH activators might additionally polarize microglia toward anti-inflammatory M2 phenotype, thereby resulting in neuroprotection. Inflammation also plays a crucial downstream role in epilepsy.

Indeed, mounting evidence suggests that neuroinflammation is not just a consequence but an important contributor to the development and progression of epilepsy. It indicates that microglial activation and microglia-mediated inflammation exert dual (beneficial/harmful) effects in epilepsy pathophysiology. Following an initial brain insult, microglia are polarized into activated phenotypes (“M1 phenotype”) and release various factors (cytokines, chemokines, growth factors, reactive oxygen species) that if left unregulated can contribute to ongoing neuroinflammation with detrimental consequences to nearby neurons. On the other hand, “M2 activation” implicates the release of immunoregulatory or “anti-inflammatory” cytokines that may promote repair mechanisms [Hickman et al. (2013) Nature neurosci. 16, 1896; Xue et al. (2014) Immunity 40, 274-288]. Altering the balance of these microglia polarization markers to a predominantly anti-inflammatory “M2”/reparative phenotype is a novel therapeutic approach against acquired epilepsy [Therajaran et al. (2020) Epilepsia 61, 203-215].

The present invention provides idebenone as a medicament to remediate drug resistant epilepsy and neuroinflammation, thereby polarizing microglia from M1 to M2 phenotype. The latter is tested in a mouse model of temporal lobe epilepsy. The polarization of microglia towards “M2” like phenotype or at least tilting the balance in favour of “M2”, induced by idebenone, is assessed by qPCR evaluation for markers/cytokines including IL-1B, TNF, IL-6 Arginase1, PPAR-G2, YM1, IL-4, IL-13, IL-10, Dusp1 etc.

The effectiveness of a compound for use in the treatment and prevention of a drug resistant epilepsy is further assessed in a Zebrafish EKP-induced seizure model. Zebrafish have emerged as a promising new animal model for epileptic seizure disorders, with particular relevance for genetic and developmental epilepsies [Burrows et al. (2020) Eur J Paediatr Neurol. 24, 70-80]. Although it underwent a whole genome duplication, the zebrafish genome is highly homologous to the human genome, with over 80% conservation of disease-causing genes, whilst also being genetically tractable. A recent review of this literature indicates that zebrafish models of epilepsy featuring spontaneous seizures can be more reliable in terms of clinical relevance and pharmacological predictability than their mammalian counterparts [Griffin et al. (2018) Front Pharmacol. 9, 573]. Consequently, over recent years several chemical and genetic zebrafish models of acute seizures or epilepsy have been generated either by immersion of larvae in chemical proconvulsants like pentylenetetrazol (PTZ) [Afrikanova et al. (2013) PLOS One. 8, e54166. or allylglycine (AG) [Leclercq et al. (2015) Epilepsy Behav. 45, 53-63], or by knocking-down or introducing mutations in epilepsy susceptible genes including scn1lab [Dinday et al. (2015) eNeuro. 2(4), ENEURO.0068-15.2015; Zhang et al. (2015) PLOS One. 10(5), e0125898]

More specifically, in the present invention a zebrafish EKP-induced seizure model is used. In this model, the lipid-permeable glutamic acid decarboxylase (GAD)-inhibitor, Ethyl ketopentenoate (EKP), is used that induces drug-resistant seizures in zebrafish [Zhang et al. (2017) Sci Rep. 7, 7195]. GAD, converting glutamate into γ-aminobutyric acid (GABA), is a key enzyme in the dynamic regulation of neural network excitability. Clinical evidence has shown that lowered GAD activity is associated with several forms of epilepsy that are often treatment resistant [Lloyd et al. (1986) Adv Neurol. 44, 1033-1044]. In this respect, reduced GAD activity has been found in epileptic foci from patients with intractable epilepsy indicating that failure to synthetize GABA and loss of inhibitory synaptic activity may lead to epilepsy [Lloyd et al. cited above]. Furthermore, in so-called autoimmune epilepsies GAD antibodies have been detected especially in patients with focal epilepsies like drug-resistant temporal lobe epilepsy (TLE) [Errichiello et al. (2009) J Neuroimmunol 211, 120-123; Errichiello et al. (2011) Neurol Sci 32, 547-550] Hence, chemical inhibition of GAD is relevant to induce drug resistant seizures, resulting in reduced levels of GABA and increased levels of glutamate, which is the most important proconvulsant neurotransmitter.

Allylglycine (AG) is a known GAD inhibitor and was previously used to develop a zebrafish seizure model [Leclercq cited above] (ref), showing that AG reduced GABA content and as a consequence induced epileptiform activity in zebrafish larvae and mice. However, AG-induced seizures in zebrafish were often asynchronous with long latency onset. As the oxidative metabolite of AG, i.e. 2-keto-4-pentenoic acid (KPA), was proven to be a far more potent inhibitor of GAD as compared to AG, KU Leuven explored the possibility to use ethyl ketopentenoate (EKP), a lipid-permeable form of KPA to induce refractory seizures in zebrafish larvae. This zebrafish EKP-induced seizure model was validated as a reliable model for drug-resistant epilepsy [Zhang et al cited above].

The EKP-induced zebrafish epilepsy model is a validated epilepsy model and allows to identify novel AEDs with a novel mode of action, primarily focused on restoring glutamate balance and downstream glutamate signalling, thus targeting the glutaminergic system.

### EXAMPLES

### Example 1. Idebenone Induces Increased PHGDH Activity

PHGDH enzyme activity was tested using human recombinant PHGDH (BPS bioscience, 71079) and a specific colorimetric PHGDH activity kit (Biovision, K569). As a readout, absorbance at 450 nm, indicative for the amount of NADH generated, was measured over time.

The results show that idebenone induces increased enzyme activity of PHGDH (FIG. 1); this increased activity can be blocked by co-administration of a specific PHGDH inhibitor like CBR-5884 [Mullarky et al. (2016) Proc Natl Acad Sci USA 113, 1778-1783] Idebenone itself does not affect the oxido-reduction reaction toward NADH generation.

None of the anti-epilepsy drugs (AEDs) that are currently on the market induce PHGDH activity, pointing to a novel mode of action of an AED.

### Example 2. Idebenone Blocks Seizures in a Zebrafish EKP-Model for Drug Resistant Epilepsy

The EKP-induced epilepsy zebrafish model is a suitable model to investigate drug resistant epilepsy. Ethyl ketopentenoate (EKP) is a lipid-permeable GAD-inhibitor that results in increased glutamate levels and drug resistant seizures in zebrafish [Zhang et al. (2017) Sci Rep. 7, 7195]. Glutamic acid decarboxylase (GAD) which converts glutamate into GABA is a key enzyme in the dynamic regulation of neural network excitability. Clinical evidence has shown that lowered GAD activity (resulting in increased glutamate levels) is associated with several forms of epilepsy that are often treatment resistant [Lloyd et al. cited above]. The EKP-induced epilepsy zebrafish model has been validated as a model to identify drugs that can be used to treat drug resistant epilepsy [Sourbron et al. (2019) Epilepsia 60, e8-e13].

Larvae (7dpf) in 100 μl VHC were arrayed individually in a 96-well plate (tissue culture plate, at bottom, Falcon, USA) and kept in the light at 28° C. 2 hours before Idebenone was added (8.5 μM) to the larvae and afterwards the 96-well plates were placed in darkness at 28° C. for 2 hours. Just prior to tracking 100 μl of VHC or EKP stock solution was added to each well to obtain a EKP concentration of 300 μM. The plates were placed in an automated video tracking device (ZebraBox™ apparatus; Viewpoint, Lyon, France) and the locomotor behaviour of the larvae was monitored for 40 min in the dark at 28° C. Locomotor activity was quantified using ZebraLab™ software (Viewpoint, Lyon, France) and expressed in “actinteg” units per 5-min interval. For each larvae, 30 min of tracking data after the effect of EKP was initiated was used. The actinteg value is defined as the sum of all image pixel changes detected during the time window.

The results show that idebenone significantly reduced EKP-induced seizures (assessed as locomotor movement), indicating idebenone as a AED to treat drug resistant epilepsy.

Common to all epilepsies, seizures occur due to an abnormal, excessive and synchronous activity of large neuronal populations. As such, seizures can be observed on electrophysiological recordings such as invasive or non-invasive local field potential (LFP) measurements, being a counterpart of electroencephalogram (EEG) performed in humans. Indeed, these techniques serve as a gold standard for epilepsy diagnosis. In the current context, LFP recordings for monitoring seizure activity can be used to validate novel zebrafish epilepsy models, as well as to evaluate the efficacy of new drug candidates.

Treatment of the zebrafish with 8.5-4.3 μM idebenone also significantly reduced EKP-induced epileptiform activity, as demonstrated by LFP recordings. This activity is comparable with the anti-seizure activity of 1 μM Perampanel in this model [Zhang et al. (2017) cited above]. The anti-seizure activity of idebenone could be blocked by co-administration of a specific PHGDH inhibitor CBR-5884 [Mullarky et al. (2016) Proc Natl Acad Sci USA 13, 1778-1783], pointing to PHGDH-dependent activity of idebenone.

### Example 3: Idebenone Alleviates Neuroinflammation in a Model of Temporal Lobe Epilepsy in c57bl/6 Mice

Eight week old male mice are allocated into four treatment groups which includes SHAM+vehicle, SHAM+Idebenone, SSSE+Vehicle, and SSSE+Idebenone. All the mice receive idebenone or vehicle for 7-days. At the end of the experiments, unilateral hippocampi are collected for gene expression analysis.

Mice are surgically implanted with three extradural screw electrodes (two served as ground/reference and one over contralateral parietal cortex as active electrode) and one bipolar stimulating electrode into the right ventral hippocampus at the following coordinates from bregma (anteroposterior: −3.00; mediolateral: −3.00; and dorsoventral: 2.80).

The bipolar electrode is connected to an Accupulser Pulse Stimulator (A310, World Precision Instruments, USA). An after-discharge threshold (ADT), defined as the minimum electrical current needed to induce an electrographic seizure exceeding ten seconds, is established by applying electrical stimulations of increasing electrical current (50 Hz, 1-second duration, 1-ms alternating current pulses) to the ventral hippocampus via the bipolar electrode. Subsequently, mice receive electrical stimulation through the bipolar electrode for (90 minutes duration, 100-ms trains of 1-ms alternating current pulses (50 Hz) at a suprathreshold current intensity (typically 10 μA above ADT). The current is interrupted every 9 minutes for a minute to confirm development of SSSE on EEG traces. At the end of 90-minute stimulation, mice are monitored for another 150 minutes following which SSSE is terminated with diazepam.

Microglia polarization via intraperitoneal injection of Idebenone. Next morning after the termination of SSSE, mice receive either Idebenone (concentration in the range of 10-200 mg/kg i.p.) or vehicle injections for one week. Idebenone is suspended in 1% gum acacia in normal saline OR 5% Arabic gum solution, and injected intraperitoneally (0.1 ml/10 g).

Tissue processing and qPCR. At the completion of treatment, animals are euthanized by lethal injection of pentobarbitone, and brains are removed and placed in ice-cold 0.1M phosphate-buffered saline (pH 7.4). Hippocampi are dissected and immediately frozen on dry ice and stored at −80ºC. mRNA is extracted using a Nucleospin RNA Plus kit (Machery-Nagel) according to the manufacturer's instructions. cDNA synthesis is performed using the Omniscript RT Kit (QIAGEN). The real-time quantitative PCR are completed using high throughput gene expression platform based on microfluidic dynamic arrays (48.48 Dynamic array IFC). All mRNA expression levels are reported as levels relative to housekeeping gene and are normalised to the values in control animals. Expression of following genes is assessed: Phgdh, Arginase, TGF beta, IL-10, PPAR-G2, GDNF, Ym1, Dusp1 and others.

### Example 4. Effects of Idebenone on Modulating Neuroinflammation in a Model of Temporal Lobe Epilepsy in C57bl/6 Mice

The present example investigates whether Idebenone has the potential to modulate neuroinflammation from an M1 to M2 phenotype. This was tested in a model of temporal lobe epilepsy, in which status epilepticus is induced upon electrical stimulation and idebenone was administered for one week @ 100 mg/kg twice daily i.p. (control animals received vehicle injections for one week). Idebenone was suspended in 5% dimethyl sulfoxide and 20% Kolliphor RH40 in 0.01M PBS. Expression of the major marker for M2 polarization, Arginase-1, was assessed via real-time quantitative PCR upon brain tissue processing. Two-way ANOVA analysis was performed to evaluate the effect of two factors including the treatment and the SSSE [self-sustained Electrical Status Epilepticus], and any interaction of the two factors.

The polarization of microglia towards “M2”-like phenotype (or at least tilting the balance in favour of “M2”) is of therapeutic value in preventing epilepsy and associated behavioural comorbidities.

Idebenone treated animals displayed a significant increase (p=0.017) in the expression of a key microglia/macrophage M2 marker, Arginase 1 when compared to the vehicle control animals (FIG. 5). The expression level of Arg1 was also affected by SSSE induction, where the SE animals displayed an increased expression of Arg1 when compared to the control animals.

### Example 5: Effect of Idebenone on Glutamate Levels

Zebrafish wild types AB were treated with EKP to induce drug-resistant epilepsy, or were genetically modified to induce Dravet pathology (scn1lab−/−). For the latter, heterozygous scn1lab+/−siblings were used as controls (which do not develop Dravet). Data are means of triplicate measurements (n=3); data of vehicle controls (AB+VHC; AB+VHC+EKP; scn1lab+/− or −/−+VHC) are means of 6 measurements (n=6).

1. Idebenone for use in the treatment or prevention of neuroinflammation in an epilepsy patent.

2. Idebenone for use according to claim 1, in the treatment or prevention of neuroinflammation in an epilepsy patient and in the treatment and prevention of a seizure in said patient.

3. Idebenone for use in the treatment or prevention according to claim 1, wherein the epilepsy is a drug resistant epilepsy.

4. Idebenone for use according to claim 3, wherein the drug resistant epilepsy is an epilepsy resistant against two or more drugs selected from the group consisting of valproate, carbamazepine, levetiracetam, lamotrigine, topiramate, briveracetam, lacosamide, perampanel, and phenobarbital.

5. A method of treating or preventing neuroinflammation associated with epilepsy, comprising the step of administering to an epilepsy patient a therapeutic effective amount of idebenone.

6. The method according to claim 5, wherein the method further treats or prevents an epileptic seizure in said patient.

7. The method according to claim 5 or 6, wherein the epilepsy is a drug resistant epilepsy.

8. The method according to claim 6, wherein the drug resistant epilepsy is an epilepsy resistant against two or more drugs selected from the group consisting of valproate, carbamazepine, levetiracetam, lamotrigine, topiramate, briveracetam, lacosamide, perampanel, and phenobarbital.
